(19) United States
(12) Patent Application Publication
(10) Pub. No.: US 2008/0038360 A1
(43) Pub. Date: Feb. 14, 2008

(54) Personal care composition

(76) Inventors: Joseph Michael Zukowski, Cincinnati, OH (US); Susan Adair Griffiths-Brophy, Middletown, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/502,866

(22) Filed: Aug. 11, 2006

(51) Int. Cl. A61K 9/14

(52) U.S. Cl. 424/490

(57) ABSTRACT

Personal care composition comprising from about 0.1% to about 15% of an emulsifying silicone elastomer; from about 1% to about 90% of an aqueous phase; and from about 0.001% to about 5% of a coated particulate comprising a core material and a coating material, wherein the coated particulate has a size of from about 5 microns to about 100 microns and comprises from about 1% to about 15%, by weight of the coated particulate, of the coating material, and wherein the coating material comprises polysiloxanes.

### FIELD OF THE INVENTION

The present invention relates to personal care compositions, and methods of use thereof, which exhibit improved stability and dispersion of coated particulates.

### BACKGROUND OF THE INVENTION

A variety of products are available to the consumer to provide skin care benefits and to counteract what many consider undesirable “signs of skin aging,” such as fine lines, wrinkles and uneven skin texture. To be most effective, some products must be applied regularly and over an extended period of time. To encourage frequent usage, it is important that the product have a pleasant appearance and feel, both prior to and after application.

Silicone elastomers are desirable for use in personal care compositions, in part because of their pleasant skin feel qualities. Particulate materials also are desirable in personal care compositions to impart a pleasant feel and appearance to the composition, and also for example to aid in providing a more uniform appearance to the skin. Whereas it may be possible to provide a more uniform appearance using cosmetic colorants and powders, one drawback of such materials is that relatively high amounts may be required to achieve the desired effect. This may result in an artificial, or “mask-like,” appearance. Another drawback is these particulates may result in highly colored compositions, which may be unsuitable for use as lotions and moisturizers.

Particulate materials other than cosmetic colorants and powders, such as interference pigments, provide an alternative to colored pigments for diminishing the appearance of imperfections. Such particulate materials may be coated to provide ease of formulation. For example, for use in emulsions comprising silicone elastomers and emulsifiers, the particulate materials may be coated with a hydrophobic material to facilitate distribution into the non-aqueous phase. Applicants have found, however, that a number of coated particulates are unsuitable for use in compositions comprising silicone emulsifiers, and result in agglomeration of the particulates (i.e. flocculation or precipitation), clumping and/or graininess, all of which result in compositions that have an unpleasant appearance and/or feel. For example, the compositions may have a non-uniform appearance in the container, produce streaks or an otherwise non-uniform appearance when spread onto skin, and/or feel rough and unpleasant to the touch.

There exists a need, therefore, for a stable skin care composition comprising silicone elastomers and non-cosmetic particulate materials which have a pleasant appearance and feel both prior to and after application.

### SUMMARY OF THE INVENTION

The present invention meets the aforementioned needs. Applicants have unexpectedly found that coated particulates having a size of from about 5 microns to about 100 microns and which are coated with from about 1% to about 15% of a polysiloxane coating material are suitable for use with silicone elastomers to provide compositions having the aforementioned properties.

The following represent some non-limiting embodiments of the present invention.

According to a first embodiment of the present invention, a stable personal care composition is provided comprising from about 0.1% to about 15% of an emulsifying silicone elastomer; from about 1% to about 90% of an aqueous phase; and from about 0.001% to about 5% of a coated particulate comprising a coating material, wherein the coated particulate has a size of from about 5 microns to about 100 microns and comprises from about 1% to about 15%, by weight of the coated particulate, of the coating material, and wherein the coating material comprises polysiloxanes.

According to another embodiment of the present invention, a method of providing a benefit to mammalian keratinous tissue in need thereof is provided, comprising the step of applying the composition of the first embodiment to keratinous tissue.

According to another embodiment of the present invention, a kit is provided, comprising the composition of the first embodiment.

### DETAILED DESCRIPTION OF THE INVENTION

The composition of the present invention may be used in skin care, cosmetic, and hair care products, non-limiting uses of which include moisturizers, conditioners, anti-aging compounds, skin lightening compounds, and combinations thereof. In one embodiment, the composition is applied to the face, neck, hands, arms and other typically exposed areas of the body. Alternatively, the composition is applied to insult-affected areas of keratinous tissue.

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. All measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified. “Micron,” as used herein, means 1×10⁻⁶ m.

Herein, “personal care composition” means compositions suitable for topical application on mammalian keratinous tissue. The personal care compositions of the present invention are understood not to include deodorant and/or antiperspirant compositions. “Skin care actives,” or “actives,” as used herein, means compounds that, when applied to the skin, provide a benefit or improvement to the skin. It is to be understood that skin care actives are useful not only for application to skin, but also to hair, nails and other mammalian keratinous tissue.

“Keratinous tissue,” as used herein, refers to keratin-containing layers disposed as the outermost protective covering of mammals which includes, but is not limited to, skin, hair, nails, cuticles, etc.

Herein, “stable” and “stability” mean a composition which is substantially unaltered in chemical state, physical homogeneity and/or color when the composition is at a temperature of from about 1° C. to about 40° C. “Stable” and “stability” also are understood to mean that the coated particulate is substantially evenly dispersed throughout the composition, where “substantially evenly dispersed” is understood to mean that particulate agglomerates are not visible, without the aid of magnification, and wherein the composition has a smooth, i.e. not grainy or rough, feel on the skin. “Substantially evenly dispersed” also may be understood to mean that when the composition is drawn, as described here, no streaks, agglomeration, and/or other signs of unevenness are apparent, without the aid of magnification.

“Spreadability,” as used herein, refers to the ability of the composition to be spread into a thin layer, for example as one would spread a composition onto the skin, by applying shear force such as wiping. Spreadability may be determined by the following method, which would be understood by one of skill in the art: Allow the composition and a TA-425TTC Spreadability Fixture, i.e. a male and a female acrylic 90° cone, having a height of 25 mm (Texture Technologies Corp.), to equilibrate to the desired temperature. Place an amount of composition into the cone, such that the top of the product level is flush with the top of the female cone in the absence of air pockets. Measure the force in g·s required for the male cone to traverse the product in the female cone, resulting in displacement of the product from the female cone, at a rate of 3 mm/s, until the distance between the lower portion of the male cone and the female cone is about 1 mm.

Herein, “drawn” means that the composition is applied onto at least the black portion of an opacity chart (Form 2A, Leneta Company of Manwah, N.J. or the equivalent thereof, of which the top half is black and the bottom half is white) and spread into a film having a thickness of approximately 0.0015 inches using a film applicator (e.g., as commercially available from BYK Gardner of Columbia, Md., or the equivalent thereof). The chroma may be measured on the black portion of the opacity chart after the film is allowed to dry for 4 hours under conditions of 22° C.+/−2° C., 1 atm using a chromameter (e.g., a Minolta CR-200 Chromameter, d65 illuminant, 0 degree viewing angle, commercially available from the Minolta Camera Co. of Ramsey, N.J. and described in the chromameter manual, version 3.0; 1988, incorporated herein by reference, or the equivalent thereof). “Keratinous tissue,” as used herein, refers to keratin-containing layers disposed as the outermost protective covering of mammals which includes, but is not limited to, skin, hair, nails, cuticles, etc.

Herein, “chroma,” describes color and color intensity. For the purposes of the present invention, color is defined according to a value on the CIELAB color system, which is based on the XYZ color system, defined by the Commission Internationale de l'Eclairage (CIE system) to provide a manner of objectively representing perceived color and color differences. X, Y and Z can be expressed in a variety of manners, or “scales,” one of which is the Hunter scale. The Hunter scale has three variables, L, a, and b, which correlate mathematically to X, Y and Z, and is described by Robertson, A. R. in “The CIE 1976 Color Difference Formulas,” Color Research Applications, vol. 2, pp. 7-11 (1977). The compositions of the present invention may be analyzed with a MINOLTA® CR-200 Chroma Meter, which generates values for L, a, and b. The value for “a” correlates to a value along the red-green (horizontal) axis, and the value for “b” correlates to a value along the blue-yellow (vertical) axis. For example, a blue-colored sample will have a negative b-value, whereas a red-colored sample will have a positive a-value. A more positive or negative value represents a more intense color. The value for “L” is an indicator of lightness and/or darkness, and correlates to a value along the z-axis, which is perpendicular to both the horizontal and vertical axes. “Chroma” is measured by a vector having its origin at the intersection of the red-green and blue-yellow axes and extending outward into the color space defined by the horizontal and vertical axes of the CIELAB color system. The length of the vector represents the chroma, and the direction of the vector represents the shade, or hue. The shorter the vector, the less colored is the composition, and the lower the chroma. Herein, “substantially colorless” used in reference to bulk compositions means the composition has a chroma of about 10.0 or less, and is understood herein to include white compositions.

Herein, “bulk” means a volume of composition, for example at least 1 cubic centimeter (ccm), which has not been spread out, or “drawn.”

Herein, “contrast ratio” refers to the opacity of the composition, or the ability of the composition to reduce or prevent light transmission, determined after the composition is drawn onto an opacity chart (Form 2A, Leneta Company of Manwah, N.J. or the equivalent thereof), and by using a chromameter (e.g., a Minolta CR-200 Chromameter, d65 illuminant, 0 degree viewing angle, commercially available from the Minolta Camera Co. of Ramsey, N.J. and described in the chromameter manual, version 3.0; 1988, incorporated herein by reference, or the equivalent thereof). The composition is drawn into a film having a thickness of approximately 0.0015 inches as described above. The film is allowed to dry for 4 hours under conditions of 22° C.+/−1° C., 1 atm. Using the chromameter, the Y tristimulus value (i.e., the XYZ color space of the film) of the product film is measured and recorded. The Y tristimulus value is measured in three different areas of the product film over the black section of the opacity chart, and also in three different areas of the product film over the white section of the opacity chart. The contrast ratio is calculated as the mathematical average of the three Y tristimulus values over the black areas, divided by the mathematical average of the three Y tristimulus values over the white areas, times 100:

Contrast Ratio=[average (three Y_{black})/average (three Y_{white})]×100.

Herein, “adjusted contrast ratio” means a contrast ratio which has been calibrated by subtracting the contrast ratio of a blank opacity chart, i.e. a chart without any product applied.

“Hardness,” as used herein, means the amount of force in grams (g) necessary for a probe having a diameter of 2 mm to penetrate a distance of 0.3 mm at a rate of 0.1 mm/s into the composition, using a TA-XT21 Texture Analyzer with Texture Expert Exceed software (v. 2.64). Prior to measuring the hardness, the composition is allowed to equilibrate to a first temperature or a second temperature, as indicated herein. When no temperature is indicated, the temperature of the composition is 25° C. All temperatures are understood to be ±1° C.

“Visibly separated,” as used herein, means that a composition in the form of an emulsion releases the aqueous phase (i.e. is “water-releasing”) upon application to the skin or other keratinous tissue. Whether a composition visibly separates may be determined by applying a constant shear rate to a water-in-oil emulsion having an average water droplet size of about 1 micron or less, by using a standard optical microscope with differential scanning contrast capabilities and a shear stage (Linkam Scientific Instruments No. 8, Epsom Downs Metro Center, Waterfield Tadworth Surrey KT20 5HT UK). Apply about 1.5 grams of the composition to the shear stage, setting a steady mode experiment having a gap width setting of 1 mm and a constant shear rate of 16 s⁻¹. With a water-releasing composition, as defined herein, an amorphous region of water having a size of from about 10 microns to at least 75 microns becomes visible at 500× magnification within about 1 minute of applying shear. Compositions that do not release when applied to the skin do not exhibit a significant change in the water droplet size when exposed to these conditions.

“Applied” or “application,” as used herein, means to spread the composition onto keratinous tissue with one or more fingers and/or an implement, using one continuous, unidirectional motion and light pressure, for example, as one would be expected to apply a cream to the facial skin.

Herein, “colorant,” or equivalent terms, refers to a substance which imparts color in a subtractive (i.e. all colors combine to produce black), rather than an additive system. Examples of colorants include, but are not limited to, dyes, organic and inorganic pigments other than non-cosmetic particulates and interference pigments, etc.

Herein, “delivery enhancement device” means any device that increases the amount of active ingredient applied to and/or into the skin relative to the amount of active ingredient that is delivered without using the device.

Herein, “regulating skin condition” means improving skin appearance and/or feel, for example, by providing a benefit, such as a smoother appearance and/or feel. Herein, “improving skin condition” means effecting a visually and/or tactilely perceptible positive change in skin appearance and feel. The benefit may be a chronic benefit and may include one or more of the following: Reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by hyperpigmentation, etc.

As used herein, “signs of skin aging,” include, but are not limited to, all outward visibly and tactilely perceptible manifestations, as well as any macro- or microeffects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; loss of skin elasticity; discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

Herein, “insult-affected keratinous tissue,” means keratinous tissue which exhibits discomfort, irritation, an unpleasant or irregular appearance and the like, for example after exposure to a physical and/or chemical irritant. Non-limiting examples of insult-affected keratinous tissue include sunburn and other types of burns; rashes, such as diaper rash, shaving rash and allergen-induced rashes; discoloration, such as bleaching, staining or hyperpigmentation; skin having nicks and cuts due to, for example, shaving; dry, chapped or rough skin due to exposure to example wind, cold and/or low humidity, etc. Non-limiting examples of insults include radiation, wind, low humidity, allergens, pollutants, chemical and natural irritants, bodily fluids, bodily waste, excessive moisture, bacteria, fungi, etc.

“Non-volatile,” as used herein, means materials that exhibit a vapor pressure of no more than about 0.2 mm Hg at 25° C. at one atmosphere and/or to materials that have a boiling point at one atmosphere of at least about 300° C. “Volatile,” as used herein, all materials that are not “non-volatile” as defined herein.

“Non-polar,” as used herein, means that the material has an average solubility parameter below about 6.5 (cal/cm³)^{0.5}, where “cal” means calories. Oils having a higher solubility parameter than 6.5 may be used if, when the oils are blended with other oils, the weighted average of the solubility parameter of the oil blend is below about 6.5. Herein, “weighted average” means that the volumes and the solubility parameters of the various oils are taken into account when calculating the average solubility parameter. “Polar,” as used herein means that the material has a higher average solubility parameter than non-polar compounds as defined herein. Solubility parameters are discussed extensively by C. D. Vaughan in “The Solubility Parameter: What is it?,” Cosmetics & Toiletries vol. 106, November, 1991, pp. 69-72, and also by C. D. Vaughan in “Using Solubility Parameters in Cosmetics Formulation”, 36 J. Soc. Cosmetic Chemists 319-333, September/October, 1988.

### I. Composition

The composition of the present invention is in the form of an emulsion and comprises a non-aqueous phase and an aqueous phase. The composition may comprise from about 10% to about 99%, alternatively from about 20% to about 90%, alternatively from about 35% to about 70%, and alternatively from about 40% to about 60%, of the non-aqueous phase. Suitable types of emulsions include, but are not limited to, oil-in-water, water-in-oil, water-in-oil-in-water, and oil-in-water-in-oil emulsions. The oil may be derived from animals, plants, or petroleum, may be natural or synthetic, and may comprise silicone oils. In one embodiment, the composition is in the form of a water-in-oil emulsion. Alternatively, the composition is a water-in-silicone emulsion.

In one embodiment, the composition comprises from about 1% to about 90%, alternatively from about 10% to about 80%, alternatively from about 30% to about 65%, and alternatively from about 40% to about 60%, of an aqueous phase. The aqueous phase may comprise from about 1% to about 70%, alternatively from about 5% to about 50%, alternatively from about 10% to about 25%, and alternatively from about 15% to about 22%, by weight of the composition, of non-water components, including but not limited to water-soluble moisturizing agents, conditioning agents, humectants and/or other water-soluble skin care actives, to impart an increased benefit to the keratinous tissue. In one embodiment, the non-water component comprises glycerin, water-soluble skin care actives, and combinations thereof. In one embodiment, the non-water component is glycerin.

The composition of the present invention has a first hardness, measured as described herein at a first temperature, and a second hardness, also measured as described herein, at a second temperature. The first hardness may be from about 2 g to about 45 g, alternatively from about 2 g to about 40 g, alternatively from about 5 g to about 35 g, alternatively from about 5 g to about 20 g, and alternatively from about 5 g to about 12 g, at a first temperature of 21° C. The second hardness, at a second temperature of 33° C., may be about 65% or less, alternatively about 55% or less, alternatively about 45% or less, and alternatively about 30% or less, of the first hardness. Alternatively, the first hardness of the composition at a first temperature of 21° C. decreases by at least 35%, and alternatively by at least 45%, alternatively by at least 55%, and alternatively by at least 70%, at a second temperature of 33° C. Alternatively, the second hardness at a second temperature of 33° C. is from about 0.1 g to about 30 g, alternatively from about 0.1 g to about 20 g, and alternatively from about 0.1 g to about 10 g, and alternatively from about 0.4 g to about 5 g.

The composition of the present invention may have a spreadability, measured as described herein, 1,000 g·s to about 10,000 g·s at a temperature of 21° C. of from about 500 g·s to about 2,500 g·s at a temperature of 33° C. In one embodiment, the spreadability of the composition at 33° C. is from about 10% to about 50%, and alternatively from about 20% to about 35%, of the spreadability of the composition at 21° C.

The composition of the present invention is stable as defined herein when the composition is at a temperature of about 40° C. In one embodiment, a composition is described which exhibits signs of instability at a temperature of above 40° C. and which, and which is fit for the originally intended use when the composition is cooled to a temperature of about 40° C. or less. For example, if the composition melts and is again cooled, the composition substantially resumes its stable form and retains desirable properties such as skin feel and appearance, and is suitable for use as described herein.

The composition may maintain rheology when hardness is reduced as described herein, at an elevated temperature. The stability of the rheology may be measured after the composition has equilibrated to a substantially uniform temperature of 45° C.±1° C. with a Brookfield™ RVDV-II+Viscometer on a Brookfield Helipath Stand equipped with a T-bar spindle (size C) rotating at 5 rpm. The viscosity may be measured at one or more points as the spindle is moved in a downward direction through previously undisturbed product. The composition may have a viscosity of from about 5,000 centipoise (cps) to about 500,000 cps, alternatively from about 10,000 cps to about 300,000 cps, alternatively from about 20,000 cps to about 200,000 cps, and alternatively from about 40,000 cps to about 140,000 cps, all at 45° C.

In one embodiment, upon application of shear force, or shear stress, the aqueous phase is visibly separated from the oil phase and the aqueous phase may coalesce to form visible droplets within and/or upon the oil phase. The oil phase typically is substantially evenly distributed upon the skin. The aqueous phase may form visible droplets immediately upon application, and alternatively within about three seconds after application, and alternatively within about ten seconds after application.

Examples of shear force include applying to the skin, or other keratinous tissue, for example by smearing, rubbing, dabbing, wiping, etc. with a finger, hand, implement and/or a delivery enhancement device. The separate aqueous phase may provide immediate benefits, including but not limited to, an immediate indication that the product is hydrating the keratinous tissue and/or an enhanced pleasant (“silky”) feel upon application. After separation of the phases, the aqueous phase may for example be rubbed into the skin or may be allowed to evaporate.

In one embodiment, the composition is substantially free of free of ester oils, wherein substantially free is understood to include an oil that is liquid at 25° C. and which comprises at least one ester moiety, for example, a monoester. Examples of ester oils are disclosed in U.S. 2006/0013792. “Substantially free of ester oils” is understood to mean that the composition comprises less than 1%, and alternatively less than 5%, of an ester oil.

In one embodiment, the bulk composition may be substantially colorless in the absence of colorants, for example, cosmetic dyes and pigments. “Substantially colorless” is understood to include white compositions. In one embodiment, the bulk composition of the present invention may have a chroma of from about 0 to about 22, alternatively from about 0 to about 12, alternatively from about 0 to about 9, and alternatively from about 0 to about 6. Additionally or alternatively, the composition of the present invention may have a chroma of from about 0 to about 8, alternatively from about 0 to about 6, alternatively from about 0 to about 3, and alternatively from about 0 to about 2, when drawn on a black surface. Additionally or alternatively, the composition of the present invention may have an adjusted contrast ratio of from about 0 to about 35, alternatively from about 0 to about 20, alternatively from about 0 to about 12, and alternatively from about 0 to about 9.

### 1. Coated Particulate

The composition of the present invention comprises a coated particulate. The composition may comprise from about 0.001% to about 25%, alternatively from about 0.001% to about 15%, alternatively from about 0.001% to about 5%, and alternatively from about 0.01% to about 2%, of the coated particulate. The coated particulate comprises a core material, or uncoated particulate, and a coating material which is covalently bonded to the core material. The coated particulate may have a size of from about 0.1 microns to about 100 microns, alternatively from about 5 microns to about 100 microns, and alternatively from about 5 microns to about 25 microns. In one embodiment, the size is from about 5 microns to about 100 microns.

The coated particulate of the present invention comprises at least 1%, and alternatively at least 5%, by total weight of the coated particulate, of the coating material. Alternatively, the coated particulate may comprise from about 1% to about 15%, alternatively from about 2% to about 10%, alternatively from about 2% to about 6%, and alternatively from about 4% to about 10% of the coating material. The coating material may comprise polysiloxanes. Non-limiting examples of suitable polysiloxane coating materials are described in U.S. Pat. No. 5,143,722, issued to Hollenberg et al. In one embodiment, the coating material comprises linear, as opposed to branched, polysiloxanes. In one embodiment, the coating material comprises methicone dimethicone, or mixtures thereof. In one embodiment, the coating material does not comprise alkyl moieties having greater than one carbon atom. Non-limiting examples of commercially available coated particulates suitable for use herein include KTZ Fine White Mica 11S2, 11S6, BAS2, BAS6, MS6, DS6, BS2, BS6, and mixtures thereof, available from KOBO.

In general, any uncoated particulate capable of being coated with at least 1% of a polysiloxane coating may be suitable for use herein. The uncoated particulate may be platelet shaped, spherical, elongated or needle-shaped, or irregularly shaped, surface coated or uncoated, porous or non-porous, charged or uncharged. In one embodiment, the uncoated particulate is platelet shaped. Non-limiting examples of suitable uncoated particulates include colored and uncolored pigments, interference pigments, inorganic powders, organic powders, composite powders, optical brightener particles, and mixtures thereof. In one embodiment, the uncoated particulate is mica, iron oxide, titanium dioxide, boron nitride, and mixtures thereof. In one embodiment, the uncoated particulate mateiral is an interference pigment. Herein, “interference pigments” means thin, platelike layered particles having two or more layers of controlled thickness. The layers have different refractive indices that yield a characteristic reflected color from the interference of typically two, but occasionally more, light reflections, from different layers of the platelike particle. Examples of interference pigments include micas layered with about 50-300 nm films of TiO₂, Fe₂O₃, silica, tin oxide, and/or Cr₂O₃. Such pigments often are pearlescent. Pearlescent pigments reflect, refract and transmit light due to the transparency of pigment particles and the large difference in the refractive index of mica platelets and, for example, the titanium dioxide coating. Intereference pigments are available commercially from a wide variety of suppliers, for example, Rona (Timiron™ and Dichrona™), Presperse (Flonac™), Englehard (Duochrome™), Kobo (SK-45-R and SK-45-G), BASF (Sicopearls™) and Eckart (Prestige™). In one embodiment, the average diameter of the longest side of the individual particles of interference pigments is less than about 75 microns, and alternatively less than about 50 microns.

Other non-limiting examples of suitable uncoated particulates can be found in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2728, and further include, but are not limited to almond meal, aluminum oxide, apricot seed powder, bismuth oxychloride, cellulose and cellulose derivatives, clay, calcium oxide, inorganic salts, for example salts of carbonates and chlorides, jojoba seed powder, loofah, peach pit powder, pecan shell powder, polyethylene, polybutylene, polyisobutylene, polymethylstyrene, polypropylene, polystyrene, polyurethane, nylon, polytetrafluoroethylene, polyhalogenated olefins, pumice, rice bran, sericite, silk, synthetic hectorite, titanium dioxide, tricalcium phosphate, zinc oxide, and mixtures thereof. Also useful are particles made from mixed polymers (e.g., copolymers, terpolymers, etc.), among such are polyethylene/polypropylene copolymer, polyethylene/propylene/isobutylene copolymer, polyethylene/styrene copolymer, and mixtures thereof. Other examples of useful particles include waxes and resins such as paraffins, carnuba wax, ozekerite wax, candellila wax, and urea-formaldehyde resins. When such waxes and resins are used herein it is important that these materials are solids at ambient and skin temperatures.

Suitable organic powders include, but are not limited, to spherical polymeric particles chosen from the methylsilsesquioxane resin microspheres, for example, Tospearl™ 145A, (Toshiba Silicone); microspheres of polymethylmethacrylates, for example, Micropearl™ M 100 (Seppic); the spherical particles of crosslinked polydimethylsiloxanes, for example, Trefil™ E 506C or Trefil™ E 505C (Dow Corning Toray Silicone); sphericle particles of polyamide, for example, nylon-12, and Orgasol™ 2002D Nat C05 (Atochem); polystyrene microspheres, for example Dyno Particles, sold under the name Dynospheres™, and ethylene acrylate copolymer, sold under the name FloBead™ EA209 (Kobo); aluminium starch octenylsuccinate, for example Dry Flo™ (National Starch); microspheres of polyethylene, for example Microthene™ FN510-00 (Equistar), silicone resin, polymethylsilsesquioxane silicone polymer, platelet shaped powder made from L-lauroyl lysine, and mixtures thereof.

In addition to coated particulates, the composition may comprise from about 0.001% to about 25% of an uncoated particulate.

### 2. Elastomers

The composition of the present invention comprises a silicone elastomer, useful for reducing the tackiness of the composition and for providing a pleasant feel upon application. One non-limiting example of useful silicone elastomers are crosslinked organopolysiloxane (or siloxane) elastomers, as described in U.S. patent publication 2003/0049212A1. The elastomers may comprise emulsifying and non-emulsifying silicone elastomers. “Emulsifying,” as used herein, means crosslinked organopolysiloxane elastomers having at least one polyoxyalkylene (e.g., polyoxyethylene or polyoxypropylene) or polyglycerin moiety, whereas “non-emulsifying” means crosslinked organopolysiloxane elastomers essentially free of polyoxyalkylene or polyglycerin moeities.

The composition of the present invention may comprise from about 0.1% to about 15%, alternatively from about 0.1% to about 5%, and alternatively from about 0.1% to about 2% of a non-emulsifying crosslinked siloxane elastomer. In one embodiment, the non-emulsifying crosslinked siloxane elastomers are dimethicone/vinyl dimethicone crosspolymers, supplied by a variety of suppliers including Dow Corning™ (DC 9040 and DC 9041), General Electric™ (SFE 839), Shin EtSu™ (KSG-15, 16, 18 [dimethicone/phenyl vinyl dimethicone crosspolymer]), and Grant Industries (GRANSIL™ line of elastomers). Cross-linked siloxane elastomers useful in the present invention and processes for making them are further described in U.S. Pat. No. 4,970,252 to Sakuta, et al.; U.S. Pat. No. 5,760,116 to Kilgour, et al.; and U.S. Pat. No. 5,654,362 to Schulz, Jr., et al. issued Aug. 5, 1997. Additional crosslinked organopolysiloxane elastomers useful in the present invention are disclosed in Japanese Patent Application JP 61-18708, assigned to Pola Kasei Kogyo KK. In addition, suitable organopolysiloxane elastomer powders include vinyl dimethicone/methicone silesquioxane crosspolymers such as KSP-100, KSP-101, KSP-102, KSP-103, KSP-104, KSP-105 (Shin Etsu™); hybrid silicone powders comprising a fluoroalkyl group, such as KSP-200 (Shin Etsu™); and hybrid silicone powders comprising a phenyl group, such as KSP-300 (Shin Etsu™) and DC-9506 (Dow Corning™).

The composition of the present invention may comprise from about 0.1% to about 15%, alternatively from about 0.2% to about 5%, and alternatively from about 0.2% to about 2% of an emulsifying crosslinked organopolysiloxane elastomer, described in U.S. Pat. Nos. 5,412,004; 5,837,793; and 5,811,487. Non-limiting examples of suitable emulsifying elastomers include polyoxyalkylene-modified elastomers formed from divinyl compounds, e.g. siloxane polymers with at least two free vinyl groups bonded via Si—H linkages on a polysiloxane backbone. In one embodiment, the emulsifying crosslinked organopolysiloxane elastomers are dimethyl polysiloxanes crosslinked by Si—H sites on a molecularly spherical MQ resin (R3SiO_{1/2} SiO_{4/2}), and alternatively is dimethicone copolyol crosspolymer and dimethicone, commercially available from Shin Etsu as KSG-21.

### 3. Elastomer Solvent

The composition of the present invention may comprise from about 1% to about 70%, alternatively from about 5% to about 55%, alternatively from about 5% to about 40%, alternatively from about 10% to about 45%, alternatively from about 20% to about 40%, and alternatively from about 1% to about 10%, of a suitable solvent for the crosslinked organopolysiloxane elastomers. Non-limiting examples of suitable solvents are described in U.S. patent publication 2003/0049212A1. The concentration of the solvent in the cosmetic compositions of the present invention may vary depending upon the type and amount of solvent and the cross-linked siloxane elastomer employed, and when combined with the cross-linked organopolysiloxane elastomer particles of the present invention, suspends and swells the elastomer particles to provide an elastic, gel-like network or matrix. The carrier for the cross-linked siloxane elastomer is liquid under ambient conditions, and in one embodiment has a low viscosity to provide for improved spreading on the skin.

The solvent may comprise volatile, non-polar oils; non-volatile, polar oils; non-volatile, non-polar oils; and non-volatile paraffinic hydrocarbon oils. Non-limiting examples of suitable non-polar, volatile oil are disclosed in U.S. Pat. No. 4,781,917 issued to Luebbe et al. and include polydecanes such as isododecane and isodecane (e.g., Permethyl-99A, available from Presperse™ Inc.) and C7-C15 isoparaffins (e.g. the Isopar Series, from Exxon™ Chemicals); cyclomethicones of varying viscosities, e.g., Dow Corning™ 200, Dow Corning™ 244, Dow Corning™ 245, Dow Corning™ 344, and Dow Corning™ 345, Silicone Fluids, commercially available from G.E. Silicones, (e.g. SF-1204, SF-1202, GE 7207 and GE 7158); and SWS-03314 (commercially available from SWS Silicones™ Corp.).

Polar, non-volatile oils useful in the present invention include, but are not limited to, silicone oils; hydrocarbon oils; fatty alcohols; fatty acids; esters of mono and dibasic carboxylic acids with mono and polyhydric alcohols; polyoxyethylenes, polyoxypropylenes, mixtures of polyoxyethylene and polyoxypropylene ethers of fatty alcohols; and mixtures thereof. In one embodiment, the polar, non-volatile oil is selected from the group consisting of propoxylated ethers of C14-C18 fatty alcohols having a degree of propoxylation below about 50, esters of C2-C8 alcohols and C12-C26 carboxylic acids (e.g. ethyl myristate, isopropyl palmitate), esters of C12-C26 alcohols and benzoic acid (e.g. Finsolv™ TN supplied by Finetex™), diesters of C2-C8 alcohols and adipic, sebacic, and phthalic acids (e.g., diisopropyl sebacate, diisopropyl adipate, di-n-butyl phthalate), polyhydric alcohol esters of C6-C26 carboxylic acids (e.g., propylene glycol dicaprate/dicaprylate, propylene glycol isostearate); and mixtures thereof.

Examples of suitable non-volatile, non-polar oils include, but are not limited to non-volatile polysiloxanes, paraffinic hydrocarbon oils, and mixtures thereof. The polysiloxanes useful in the present invention selected from the group consisting of polyalkylsiloxanes, polyarylsiloxanes, polyalkylarylsiloxanes, poly-ethersiloxane copolymers, and mixtures thereof. Examples of useful oils include Viscasil™ series (General Electric); the Dow Corning 200 series (Dow Corning Corp.); SF 1075 methyl-phenyl fluid (General Electric) and 556 Cosmetic Grade Fluid (Dow Corning Corp.).

Non-volatile paraffinic hydrocarbon oils useful in the present invention are described in U.S. Pat. No. 5,019,375 issued to Tanner et al. and in 2003/0049212A1, and include mineral oils and branched-chain hydrocarbons such as Permethyl™ 102A, 103A and 104A (Permethyl Corporation); and Ethylflo™ 364 (Ethyl Corp.). Additional suitable solvents useful herein are described in U.S. Pat. No. 5,750,096 to Guskey et al.

### 4. Emulsifier

The composition of the present invention may contain an additional emulsifier, useful for dispersing and suspending the aqueous phase within the oil phase in a water-in-oil emulsion. The composition may comprise from about 0.001% to about 5%, alternatively from about 0.01% to about 3% alternatively from about 0.1% to about 2%, and alternatively from about 0.1% to about 1%, of at least one additional emulsifier. Applicants have found that when present in amounts greater than the aforementioned amounts, the compositions may have an unpleasant skin feel, such as tackiness, or other undesirable characteristics.

A wide variety of emulsifying agents can be employed herein to form a water-in-silicone emulsion, and are described in U.S. patent publication 2003/0049212A1. In one embodiment, the additional emulsifiers are silicone emulsifiers, including organically modified organopolysiloxanes (silicone surfactants) such as dimethicone copolyols. Examples of commercially available dimethicone copolyols useful herein are Dow Corning® 190, 193, Q2-5220, 2501 Wax, 2-5324 fluid, and 3225C; ABIL™ EM-90, ABIL WE-09 and ABIL® WS-08 (Goldschmidt), KF-6028 and KF-6106 (Shin-Etsu™).

In one embodiment, the additional emulsifier is a non-silicone emulsifier, non-limiting examples of which include non-ionic and anionic emulsifying agents such as sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C₁-C30 fatty acids, C1-C30 esters of polyols, C1-C30 ethers of polyols, alkyl phosphates, polyoxyalkylene fatty ether phosphates, fatty acid amides, acyl lactylates, soaps, and mixtures thereof.

### 5. Solidifying Agent

The composition of the present invention comprises one or more solidifying agents suitable to impart stability to the composition at a temperature of about 40° C. and to impart a suitable hardness as described herein. A variety of suitable solidifying agents may be used, including those disclosed in U.S. Pat. No. 6,696,049, issued to Vatter et al. In one embodiment, the solidifying agent is a wax. The composition may comprise from about 0.1% to about 20%, alternatively from about 0.5% to about 15%, alternatively from about 1% to about 5%, and alternatively from about 5% to about 15%, of one or more solidifying agents.

Waxes suitable for use herein include but are not limited to animal, vegetable, mineral, or silicone waxes. Generally such waxes have a melting point ranging from about 25° C. to 125° C., and alternatively from about 30° C. to about 100° C. Non-limiting examples of suitable waxes include silicone waxes, fatty esters, for example cetyl and/or stearyl esters, acacia, beeswax, ceresin, flower wax, citrus wax, carnauba wax, jojoba wax, japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, polyalkylene glycol derivatives thereof (for example PEG6-20 beeswax, or PEG-12 carnauba wax) and mixtures of any of the aforementioned waxes. In one embodiment, the wax is a polyethylene wax, and alternatively is a polyethylene wax having a melting point of less than 120° C., alternatively less than 95C, and alternatively less than 85° C.

Non-limiting examples of suitable silicone waxes are disclosed in U.S. Pat. Nos. 5,413,781 and 5,725,845, and further include alkylmethyl polysiloxanes, C10-C60 alkyl dimethicones, and mixtures thereof. Alternatively, the silicone wax may be a C16-C28 alkyl dimethicone wax. Other suitable silicone waxes include, but are not limited to stearoxydimethicone, behenoxy dimethicone, stearyl dimethicone, cetearyl dimethicone, cetyl dimethicone, and mixtures thereof.

### 6. Colorants

The composition may comprise from about 0.0001% to about 2%, alternatively from about 0.001% to about 1%, and alternatively from about 0.001% to about 0.25%, of a colorant, including dyes and pigments other than the coated particulates. Non-limiting examples of colorants include inorganic pigments, such as iron oxides, ferric ammonium ferrocyanide, manganese violet, ultramarine blue, and chromium oxide. Organic pigments may include natural colorants and synthetic monomeric and polymeric colorants, for example phthalocyanine blue and green pigment. Also useful are lakes, primary FD&C or D&C lakes and blends thereof; encapsulated soluble or insoluble dyes and other colorants; and mixtures of any of the foregoing. Alternatively, the composition may be substantially free of colorants, where “substantially free” is understood to include less than 0.001% of a colorant.

### 7. Actives

The composition of the present invention may comprise at least one skin care active (“active”), useful for regulating and/or improving the condition of mammalian skin. The active may be soluble in oil or water, and may be present primarily in the oil phase and/or in the aqueous phase. Solubility in water and oil is within the knowledge of one of skill in the art, and can be determined using known methods of analysis. One of skill in the art further will understand that solubility may be affected by the type and concentration of other components in the composition, and other conditions such as pH, ionic strength, etc. Many skin care actives may provide more than one benefit, or operate via more than one mode of action; therefore, classifications herein are made for the sake of convenience and are not intended to limit the active to that particular application or applications listed.

### Vitamins

The compositions of the present invention may comprise from about 0.0001% to about 50%, alternatively from about 0.001% to about 10%, alternatively from about 0.01% to about 5%, of at least one vitamin. Herein, “vitamins” means vitamins, pro-vitamins, and their salts, isomers and derivatives. Non-limiting examples of suitable vitamins include: vitamin B compounds (including B1 compounds, B2 compounds, B3 compounds such as niacinamide, niacinnicotinic acid, tocopheryl nicotinate, C1-C18 nicotinic acid esters, and nicotinyl alcohol; B5 compounds, such as panthenol or “pro-B5”, pantothenic acid, pantothenyl; B6 compounds, such as pyroxidine, pyridoxal, pyridoxamine; carnitine, thiamine, riboflavin); vitamin A compounds, and all natural and/or synthetic analogs of Vitamin A, including retinoids, retinol, retinyl acetate, retinyl palmitate, retinoic acid, retinaldehyde, retinyl propionate, carotenoids (pro-vitamin A), and other compounds which possess the biological activity of Vitamin A; vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids. In one embodiment, the composition comprises a vitamin selected from the group consisting of vitamin B compounds, vitamin C compounds, vitamin E compounds and mixtures thereof. Alternatively, the vitamin is selected from the group consisting of niacinamide, tocopheryl nicotinate, pyroxidine, panthenol, vitamin E, vitamin E acetate, ascorbyl phosphates, ascorbyl glucoside, and mixtures thereof.

### Peptides and Peptide Derivatives

The compositions of the present invention may comprise one or more peptides. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, carnosine (beta-alanine-histidine), palmitoyl-lysine-threonine (pal-KT) and palmitoyl-lysine-threonine-threonine-lysine-serine (pal-KTTKS, available in a composition known as MATRIXYL), palmitoyl-glycine-glutamine-proline-arginine (pal-GQPR, available in a composition known as RIGIN®), these three being available from Sederma, France, acetyl-glutamate-glutamate-methionine-glutamine-arginine-arginine (Ac-EEMQRR; Argireline®), and Cu-histidine-glycine-glycine (Cu-HGG, also known as IAMIN®).

The compositions may comprise from about 1×10⁻⁷% to about 20%, alternatively from about 1×10⁻⁶% to about 10%, and alternatively from about 1×10⁻⁵% to about 5% of the peptide.

### Sugar Amines

The compositions of the present invention may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials). For example, glucosamine is generally found in many shellfish and can also be derived from fungal sources. Sugar amine compounds useful in the present invention include, for example, N-acetyl-glucosamine, and also those described in PCT Publication WO 02/076423 and U.S. Pat. No. 6,159,485, issued to Yu, et al. In one embodiment, the composition comprises from about 0.01% to about 15%, alternatively from about 0.1% to about 10%, and alternatively from about 0.5% to about 5%, of the sugar amine.

### Sunscreens

The compositions of the subject invention may comprise one or more sunscreen actives (or sunscreen agents) and/or ultraviolet light absorbers. Herein, “sunscreen active” includes both sunscreen agents and physical sunblocks. Sunscreen actives and ultraviolet light absorbers may be organic or inorganic. Examples of suitable sunscreen actives and ultraviolet light absorbers are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93, and further include terephthalylidene dicamphor sulfonic acid, (Mexoryl™ SX).

### Oil Control Agents

The compositions of the present invention may comprise one or more compounds useful for regulating the production of skin oil, or sebum, and for improving the appearance of oily skin. Examples of suitable oil control agents include salicylic acid, dehydroacetic acid, benzoyl peroxide, vitamin B3 compounds (for example, niacinamide or tocopheryl nicotinate), their isomers, esters, salts and derivatives, and mixtures thereof. The compositions may comprise from about 0.0001% to about 15%, alternatively from about 0.01% to about 10%, alternatively from about 0.1% to about 5%, and alternatively from about 0.2% to about 2%, of an oil control agent.

### Other Skin Care Actives

The compositions of the present invention further may comprise non-vitamin antioxidants and radical scavengers, hair growth regulators, flavonoids, minerals, preservatives, phytosterols and/or plant hormones, protease inhibitors, tyrosinase inhibitors, anti-inflammatory agents and N-acyl amino acid compounds.

Suitable non-vitamin antioxidants and radical scavengers include, but are not limited to, BHT (butylated hydroxy toluene), L-ergothioneine (available as THIOTANE™); tetrahydrocurcumin, cetyl pyridinium chloride, carnosine, diethylhexyl syrinylidene malonate (available as OXYNEX™), hexadec-8-ene-1,16-dicarboxylic acid (octadecene dioic acid; ARLATONE™ Dioic DCA from Uniqema), ubiquinone (co-enzyme Q10), tea extracts including green tea extract, yeast extracts or yeast culture fluid (e.g., Piteraφ), and combinations thereof.

Suitable hair growth regulators include, but are not limited to, hexamidine compounds, butylated hydroxytoluene (BHT), hexanediol, panthenol and pantothenic acid derivates, their isomers, salts and derivatives, and mixtures thereof.

Suitable minerals include zinc, manganese, magnesium, copper, iron, selenium and other mineral supplements. “Mineral” is understood to include minerals in various oxidation states, mineral complexes, salts, derivatives, and combinations thereof.

Suitable examples of plant sterols (phytosterols) and/or plant hormones include, but are not limited to, sitosterol, stigmasterol, campesterol, brassicasterol, kinetin, zeatin, and mixtures thereof.

Suitable protease inhibitors include, but are not limited to, hexamidine compounds, vanillin acetate, menthyl anthranilate, soybean trypsin inhibitor, Bowman-Birk inhibitor, and mixtures thereof.

Suitable tyrosinase inhibitors include, but are not limited to, sinablanca (mustard seed extract), tetrahydrocurcumin, cetyl pyridinium chloride, and mixtures thereof.

Suitable anti-inflammatory agents include, but are not limited to, glycyrrhizic acid (also known as glycyrrhizin, glycyrrhixinic acid, and glycyrrhetinic acid glycoside), glycyrrhetenic acid, other licorice extracts, and combinations thereof.

Suitable N-acyl amino acid compounds include, but are not limited to, N-acyl phenylalanine, N-acyl tyrosine, their isomers, including their D and L isomers, salts, derivatives, and mixtures thereof. An example of a suitable N-acyl amino acid is N-undecylenoyl-L-phenylalanine is commercially available under the tradename SEPIWHITE® from Seppic (France).

Other useful skin care actives include moisturizing and/or conditioning agents, such as glycerol, petrolatum, caffeine, and urea; yeast extracts (for example, Pitera™); dehydroepiandrosterone (DHEA), its analogs and derivatives; exfoliating agents, including alpha- and beta-hydroxyacids, alpha-keto acids, glycolic acid and octanoyl salicylate; antimicrobial agents; antidandruff agents such as piroctone olamine, 3,4,4′-trichlorocarbanilide (trichlosan), triclocarban and zinc pyrithione; dimethyl aminoethanol (DMAE); creatine; skin lightening agents such as kojic acid, mulberry extract, hydroquinone, arbutin, and deoxy-arbutin; (sunless) tanning agents, such as dihydroxy acetone (DHA); isomers, salts, and derivatives of any of the foregoing; and mixtures thereof.

### II. Methods of Use

The present invention describes a method of regulating and/or improving the condition of mammalian skin. The method comprises the step of topically applying to mammalian skin a personal care composition described herein. Alternatively, the method may comprise the step of applying the composition described herein to insult-affected keratinous tissue, to regulate and/or improve the condition of such tissue, and/or to provide relief from the effects of the insult. When the aqueous phase visibly separates from the oil phase upon application to the keratinous tissue, the composition may signal an immediate, or acute, benefit to a consumer and increase the penetration of water soluble skin care actives into the keratinous tissue.

The composition may be applied to any keratinous tissue, including keratinous tissue in need of one or more benefits. Benefits include regulating and/or improving the condition of keratinous tissue, non-limiting examples of which include reducing the appearance of wrinkles, reducing the appearance of deep lines, reducing the appearance of fine lines, reducing the appearance of large pores, reducing the thickness of keratinous tissue, increasing the convolution of the dermal-epidermal border, increasing elasticity, reducing the appearance of cellulite, reducing the appearance of discoloration, reducing the appearance of hyperpigmentation, reducing the appearance of under-eye circles, reducing the appearance of sallowness, and combinations thereof. Alternatively, the benefit may include reducing wrinkles, reducing deep lines, reducing fine lines, reducing large pores, reducing cellulite, reducing hyperpigmentation, reducing undereye circles, reducing puffiness, and combinations thereof.

The composition may be applied by a variety of means, including by rubbing, wiping or dabbing with hands or fingers, or by means of an implement and/or delivery enhancement device. Non-limiting examples of implements include a sponge or sponge-tipped applicator, a swab (for example, a cotton-tipped swab), a pen optionally comprising a foam or sponge applicator, a brush, a wipe, and combinations thereof. Non-limiting examples of delivery enhancement devices include mechanical, electrical, ultrasonic and/or other energy devices. In one embodiment, the composition is gently spread onto the skin to facilitate the separation of the aqueous phase from the oil-phase. When the aqueous phase has separated and coalesced into visibly enhanced droplets, the composition may be left as is on the keratinous tissue. Alternatively, the composition allowed to remain on the skin for 5 seconds, 10 seconds, 30 seconds, or 1 minute prior to being rubbed into the keratinous tissue.

The amount of the composition applied, the frequency of application and the period of use will vary widely depending upon the level of components of a given composition and the level of regulation desired. For example, from about 0.1 mg composition/cm² to about 50 mg composition/cm², and alternatively about 2 mg composition/cm² of keratinous tissue may be applied. In one embodiment, the composition is applied prior to exposure of the skin to ultraviolet radiation, and alternatively at least once daily, where “daily” and “days” mean a 24-hour period. The composition further may be applied as part of a treatment regimen, for example, once daily for 30 consecutive days, alternatively for 14 consecutive days, alternatively for 7 consecutive days and alternatively for 2 consecutive days.

The method may comprise the step of inducing a temperature change in the composition and/or in the keratinous tissue either simultaneously or sequentially with the step of applying the composition. The method further may comprise additional steps which form part of a treatment or application regimen, including the steps of applying at least one additional composition, ingesting one or more dietary supplements, cleansing, etc.

### III. Kit

The present invention further provides a kit comprising at least one composition described herein. The kit may comprise an outer packaging unit, which in turn may comprise one or more inner packaging units. In one embodiment, at least a portion of all packaging is transparent or translucent, such that the composition is visible to a consumer. One non-limiting example of a suitable outer container is a box or a tray, suitable for holding a sufficient number of inner packaging units for an indicated application regimen, for example, one application per day for one month. Alternatively, the tray may contain an array of individual inner packaging units which are organized to correspond to an indicated application regimen. The kit further may comprise an implement, which may be suitable for targeted delivery of the composition to a desired area of keratinous tissue. The composition may be packaged separately from the implement, or may be contained within the implement. The kit further may comprise a plurality of components, including one or more additional compositions, one or more orally ingestible dietary supplements, an additional implement, an additional delivery enhancement device, a temperature change element, a substrate, instructions for complying with suitable application regimens, and combinations thereof.

### EXAMPLES 1-9

The following are non-limiting examples of compositions that may be applied to keratinous tissue in accordance with the methods described herein.

| | Example | | | | |
|---|---|---|---|---|---|
| | 1 | 2 | 3 | 4 | 5 |
| Phase A | | | | | |
| DC-9040¹ | 3.00 | 3.00 | 3.00 | 3.00 | 3.00 |
| Dimethicone | 4.00 | 4.00 | 4.00 | 4.00 | 4.00 |
| Polymethylsilsesquioxane² | 4.00 | 4.00 | 4.00 | 4.00 | 4.00 |
| Cyclomethicone | 3.85 | 3.85 | 3.85 | 3.85 | 3.85 |
| KSG-210³ | 2.75 | 2.75 | 2.75 | 2.75 | 2.75 |
| Polyethylene wax⁴ | | | | | |
| DC-2503 Cosmetic Wax⁵ | | | | | |
| Fragrance | 0.10 | 0.10 | 0.10 | 0.10 | 0.10 |
| Titanium Dioxide coated with 5% | | | | | |
| dimethicone⁶ | | | | | |
| Iron Oxide coated mica coated with 6% | | | | | |
| methicone⁷ | | | | | |
| Untreated Titanium Dioxide coated mica⁸ | 1.30 | | | | |
| Titanium Dioxide coated mica coated with | | 1.30 | | | |
| 2% Triethoxycaprylsilane⁹ | | | | | |
| (Titanium Dioxide coated mica coated with | | | 1.30 | | |
| 6% Triethoxycaprylsilane¹⁰ | | | | | |
| Titanium Dioxide coated mica) coated with | | | | 1.30 | |
| 6% dimethicone¹¹ | | | | | |
| Titanium Dioxide coated mica coated with | | | | | 1.30 |
| 6% methicone¹² | | | | | |
| Phase B | | | | | |
| Glycerin | 10.00 | 10.00 | 10.00 | 10.00 | 10.00 |
| Dexpanthenol | 0.50 | 0.50 | 0.50 | 0.50 | 0.50 |
| Pentylene Glycol | 3.00 | 3.00 | 3.00 | 3.00 | 3.00 |
| Hexamidine Diisethionate¹³ | 0.10 | 0.10 | 0.10 | 0.10 | 0.10 |
| Niacinamide¹⁴ | 5.00 | 5.00 | 5.00 | 5.00 | 5.00 |
| Methylparaben | 0.20 | 0.20 | 0.20 | 0.20 | 0.20 |
| Ethylparaben | 0.05 | 0.05 | 0.05 | 0.05 | 0.05 |
| Sodium Citrate | 0.20 | 0.20 | 0.20 | 0.20 | 0.20 |
| Citric Acid | 0.03 | 0.03 | 0.03 | 0.03 | 0.03 |
| Sodium Benzoate | 0.07 | 0.07 | 0.07 | 0.07 | 0.07 |
| Sodium Chloride | 0.50 | 0.50 | 0.50 | 0.50 | 0.50 |
| FD&C Red #40 (1%) | | | | | |
| Water | q.s to 100 | q.s to | q.s to 100 | q.s to | q.s to |
| | | 100 | | 100 | 100 |
| Graininess | Unstable¹⁴ | Yes | Yes | No | No |

| | Example | | | |
|---|---|---|---|---|
| | 6 | 7 | 8 | 9 |
| Phase A | | | | |
| DC-9040¹ | 8.60 | 3.00 | 37.00 | 5.00 |
| Dimethicone | 4.09 | 4.00 | 4.00 | 4.00 |
| Polymethylsilsesquioxane² | 4.09 | 4.00 | 10.00 | 4.00 |
| Cyclomethicone | 11.43 | 0.50 | 8.22 | 11.33 |
| KSG-210³ | 5.37 | 5.25 | 2.75 | 5.40 |
| Polyethylene wax⁴ | 3.54 | | 2.41 | 2.05 |
| DC-2503 Cosmetic Wax⁵ | 7.08 | 10.00 | 4.82 | 3.77 |
| Fragrance | 0.10 | 0.10 | 0.10 | 0.10 |
| Titanium Dioxide coated with 5% dimethicone⁶ | | | | 0.35 |
| Iron Oxide coated mica coated with 6% | | | | 0.65 |
| methicone⁷ | | | | |
| Untreated Titanium Dioxide coated mica⁸ | | | | |
| Titanium Dioxide coated mica coated with 2% | | | | |
| Triethoxycaprylsilane⁹ | | | | |
| (Titanium Dioxide coated mica coated with 6% | | | | |
| Triethoxycaprylsilane¹⁰ | | | | |
| Titanium Dioxide coated mica) coated with 6% | | | | |
| dimethicone¹¹ | | | | |
| Titanium Dioxide coated mica coated with 6% | 5.00 | 0.01 | 1.00 | |
| methicone¹² | | | | |
| Phase B | | | | |
| Glycerin | 10.00 | 10.00 | 10.00 | 10.00 |
| Dexpanthenol | 0.50 | 0.50 | 0.50 | 0.50 |
| Pentylene Glycol | 3.00 | 3.00 | 3.00 | 3.00 |
| Hexamidine Diisethionate¹³ | 0.10 | 0.10 | 0.10 | 0.10 |
| Niacinamide¹⁴ | 5.00 | 5.00 | 5.00 | 5.00 |
| Methylparaben | 0.20 | 0.20 | 0.20 | 0.20 |
| Ethylparaben | 0.05 | 0.05 | 0.05 | 0.05 |
| Sodium Citrate | 0.20 | 0.20 | 0.20 | 0.20 |
| Citric Acid | 0.03 | 0.03 | 0.03 | 0.03 |
| Sodium Benzoate | 0.05 | 0.05 | 0.05 | 0.05 |
| Sodium Chloride | 0.50 | 0.50 | 0.50 | 0.50 |
| FD&C Red #40 (1%) | 0.05 | 0.05 | 0.05 | 0.05 |
| Water | q.s to 100 | q.s to 100 | q.s to 100 | q.s to 100 |
| Graininess | No | No | No | No |

¹12.5% Dimethicone Crosspolymer in Cyclopentasiloxane. Available from Dow Corning ™.
²E.g., Tospearl ™ 145A or Tospearl ™ 2000. Available from GE Toshiba ™ Silicone
³25% Dimethicone PEG-10/15 Crosspolymer in Dimethicone. Available from Shin-Etsu ™.
⁴e.g. Jeenate ™ 3H polyethylene wax from Jeen ™
⁵Stearyl Dimethicone. Available from Dow Corning ™.
⁶SAT-T-CR50 from US Cosmetics ™.
⁷KTZ Aurban Coral MS6 from Kobo Products, Inc ™.
⁸KTZ Fine White from Kobo Products, Inc ™.
⁹KTZ Fine White 11S2 from Kobo Products, Inc ™.
¹⁰KTZ Fine White 11S6 from Kobo Products, Inc ™.
¹¹KTZ Fine White DS6 from Kobo Products, Inc ™.
¹²KTZ Fine White MS6 from Kobo Products, Inc ™.
¹³Hexamidine diisethionate, availabile from Laboratoires Serobiologiques.
¹⁴Additionally or alternatively, the composition may comprise one or more other skin care actives, their salts and derivatives, as disclosed herein, in amounts also disclosed herein as would be deemed suitable by one of skill in the art.
¹⁵Unstable emulsion.

For examples 1-5, in a suitable container, combine the ingredients of Phase A. In a separate suitable container, combine the ingredients of Phase B. Mixing each phase using a suitable mixer (e.g., Anchor blade, propeller blade, IKA T25) until each is homogenous. Slowly add Phase B to Phase A while continuing to mix Phase A. Continue mixing until batch is uniform. Pour product into suitable containers and store at room temperature.

For examples 6-9, in a suitable container, combine the ingredients of Phase A. In a separate suitable container, combine the ingredients of Phase B. Heat each phase to 73 C-78° C. while mixing each phase using a suitable mixer (e.g., Anchor blade, propeller blade, IKA T25) until each reaches temperature and is homogenous. Slowly add Phase B to Phase A while continuing to mix Phase A. Continue mixing until batch is uniform. Pour product into suitable containers at 73-78° C. and store at room temperature. Alternatively, continuing to stir the mixture as temperature decreases results in lower observed hardness values at 21° C. and 33° C.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a) from about 0.1% to about 15% of an emulsifying silicone elastomer;
b) from about 1% to about 90% of an aqueous phase; and
c) from about 0.001% to about 5% of a coated particulate comprising a core material and a coating material, wherein the coated particulate has a size of from about 5 microns to about 100 microns and comprises from about 1% to about 15%, by weight of the coated particulate, of the coating material, and wherein the coating material comprises polysiloxanes.

2. The composition of claim 1, wherein the polysiloxanes are linear polysiloxanes.

3. The composition of claim 1 wherein the coating material comprises methicone, dimethicone, or combinations thereof.

4. The composition of claim 1, wherein the core material is selected from the group consisting of mica, iron oxide, titanium dioxide, boron nitride, interference pigments, and mixtures thereof.

5. The composition of claim 4 wherein the core material is an interference pigment.

6. The composition of claim 1 wherein the core material is platelet-shaped.

7. The composition of claim 1, further comprising from about 0.0001% to about 2% of a colorant.

8. The composition of claim 1, further comprising from about 0.001% to about 5% of an additional emulsifier.

9. The composition of claim 1, further comprising from about 0.1% to about 15% of a non-emulsifiying silicone elastomer.

10. The composition of claim 1, further comprising at least one solidifying agent.

11. The composition of claim 10, wherein the solidifying agent is a wax.

12. The composition of claim 11, wherein the wax is selected from the group consisting of a silicone wax, a polyethylene wax, and mixtures thereof.

13. The composition of claim 12, wherein the wax is a silicone wax.

14. The composition of claim 11, wherein the wax is selected from the group consisting of C16 to C28 alkyl dimethicone waxes, polythethylene waxes with a melting point of less than 120° C., and combinations thereof.

15. The composition of claim 10, wherein the solidifying agent comprises from about 0.1% to about 20% of a first wax having a first melting point of from about 21° C. to about 40° C., and from about 0.1% to about 20% of a second wax having a second melting point of from about 40° C. to about 120° C.

16. The composition of claim 10, wherein the composition has a first hardness at a first temperature of 21° C., and a second hardness at a second temperature of 33° C., wherein the second hardness is less than 55% of the first hardness.

17. The composition of claim 16, wherein the first hardness at the first temperature of 21° C. is from about 2 g to about 45 g and the second hardness at the second temperature of 33° C. is about 0.1 g to about 30 g.

18. The composition of claim 1 wherein the aqueous phase comprises from about 30% to about 90%, by weight of the composition, of water.

19. The composition of claim 1, wherein the aqueous phase comprises from about 0.1% to about 70% of a non-water component.

20. The composition of claim 19, wherein the non-water component is glycerin.

21. The composition of claim 1, wherein the aqueous phase visibly separates from the non-aqueous phase upon the application of shear force.

22. The composition of claim 1, wherein the adjusted contrast ratio is from about 0 to about 35.

23. The composition of claim 1 wherein the chroma of the bulk composition is from about 0 to about 22.

24. The composition of claim 1, further comprising at least one additional skin care active.

25. The composition of claim 24, wherein said additional skin care active is selected from the group consisting of vitamin B compounds, vitamin C compounds, vitamin E compounds, peptides, sugar amines, oil control agents, protease inhibitors, skin lightening agents, hexamidine compounds, and combinations thereof.

26. The composition of claim 24, wherein said skin care active is selected from the group consisting of niacinamide, palmitoyl-lysine-threonine, hexamidine diisethionate, palmitoyl-lysine-threonine-threonine-lysine-serine, N-acetyl-D-glucosamine, salicylic acid, dehydroacetic acid, sodium dehydroacetate, and combinations thereof.

27. A method of providing a benefit to mammalian keratinous tissue, comprising the step of topically applying to mammalian keratinous tissue a personal care composition comprising:
a) from about 0.1% to about 15% of an emulsifying silicone elastomer;
b) from about 1% to about 90% of an aqueous phase; and
c) from about 0.001% to about 5% of a coated particulate comprising a core material and a coating material, wherein the coated particulate has a size of from about 5 microns to about 100 microns and comprises from about 1% to about 15%, by weight of the coated particulate, of the coating material, and wherein the coating material comprises polysiloxanes.

28. The method of claim 27, wherein the mammalian keratinous tissue is insult-affected mammalian skin.

29. A kit comprising:
a) a personal care composition comprising:
i. from about 0.1% to about 15% of an emulsifying silicone elastomer;
ii. from about 1% to about 90% of an aqueous phase; and
iii. from about 0.001% to about 5% of a coated particulate comprising a core material and a coating material, wherein the coated particulate has a size of from about 5 microns to about 100 microns and comprises from about 1% to about 15%, by weight of the coated particulate, of the coating material, and wherein the coating material comprises polysiloxanes.
b) at least one additional component selected from the group consisting of at least one additional composition, at least one orally ingestible dietary supplement, an implement, a delivery enhancement device, a temperature change element, instructions for complying with suitable application regimens, and combinations thereof; and
c) instructions for complying with a regimen to provide a benefit to keratinous tissue.
